(19) United States
(12) Patent Application Publication
(10) Pub. No.: US 2023/0165700 A1
(43) Pub. Date: Jun. 1, 2023

(54) SKULL BLOCKER

(71) Applicant: Sara Gomez, La Habra, CA (US)

(72) Inventor: Sara Gomez, La Habra, CA (US)

(21) Appl. No.: 17/535,977

(22) Filed: Nov. 26, 2021

(51) Int. Cl. A61F 5/30; A61F 5/37; A61M 21/02

(52) U.S. Cl. CPC A61F 5/30; A61F 5/3707; A61M 21/02; A61M 2021/0016

(57) ABSTRACT

An illustrated view of an exemplary skull blocker for protecting a person that has entered into a self-harming event is presented. The skull blocker is useful for preventing injury to the person during a self-harming event and to diffuse or allow the event to conclude without harm to the person. Furthermore, the skull blocker to be light weight, soft, reusable and easy to use. The skull blocker provides a calming effect and distracts the afflicted person from inflicting harm upon themselves. The skull blocker further provides data for tracking impact events as well as reduces fatigue on any staff that is trying to hold or prevent the self-harming event.

### FIELD OF THE INVENTION

This invention relates to a person harming themselves. More particularly, it relates to a protective device for protecting the person from harming themself.

### BACKGROUND

Self-harm or self-injury is intentional direct injury of one's own skin tissues usually without a suicidal intention. Other terms such as biting, head banging, cutting and self-mutilation have been used for any self-harming behavior regardless of suicidal intent. The most common form of self-harm is using a sharp object to cut the skin. Other forms include scratching, hitting, or burning body parts. While earlier usage included interfering with wound healing, excessive skin-picking, hair-pulling, and the ingestion of toxins, current usage distinguishes these behaviors from self-harm. Likewise, tissue damage from drug abuse or eating disorders is not considered self-harm because it is ordinarily an unintended side-effect.

Although self-harm is by definition non-suicidal, it may still be life-threatening. People who do self-harm are more likely to commit suicide, and self-harm is found in 40-60% of suicides. Still, only a minority of self-harmers are suicidal.

The desire to self-harm is a common symptom of some personality disorders. People with other mental disorders may also self-harm, including those with depression, anxiety disorders, substance abuse, eating disorders, post-traumatic stress disorder, schizophrenia, and dissociative disorders. Neurological and developmental disorders such as autism and intellectual disability may also lead to self-harm. Studies also provide strong support for a self-punishment function, and modest evidence for anti-dissociation, interpersonal-influence, anti-suicide, sensation-seeking, and interpersonal boundaries functions. Self-harm can also occur in high-functioning individuals who have no underlying mental health diagnosis. The motivations for self-harm vary. Some use it as a coping mechanism to provide temporary relief of intense feelings such as anxiety, depression, stress, emotional numbness, or a sense of failure. Self-harm is often associated with a history of trauma, including emotional and sexual abuse. There are a number of different methods that can be used to treat self-harm and which concentrate on either treating the underlying causes or on treating the behavior itself. Other approaches involve avoidance techniques, which focus on keeping the individual occupied with other activities, or replacing the act of self-harm with safer methods that do not lead to permanent damage.

A shield is a piece of personal armor held in the hand, which may or may not be strapped to the wrist or forearm. Shields are used to intercept specific attacks, whether from close-ranged weaponry or projectiles such as arrows, by means of active blocks, as well as to provide passive protection by closing one or more lines of engagement during combat.

When a person is being physically harmful to themselves by hitting their own head, pulling out their hair, scratching their face or any other self-directed harm, staff has few if any techniques that can help prevent the self-harm. This can allow the person to inflict harm upon themselves. This can cause serious injuries, both physical and mental, as well as harm to others. Often times when the person is striking or attacking their own head, the trauma that can be caused has long term effects.

No devices are currently available that can help when a person becomes harmful to themselves other than placing the person in a strait jacket which is not optimal.

Accordingly, and in light of the foregoing, it would be desirable to devise a skull blocker that can be used during a self-harming event to diffuse or allow the event to conclude without harm to the person. It would also be advantageous for the skull blocker to be light weight, soft, reusable and easy to use.

### BRIEF DESCRIPTION OF THE DRAWINGS

FIG. 1A is an illustrated view of an exemplary skull blocker.

FIG. 1B is an illustrated view of a control panel of the skull blocker shown in FIG. 1A.

### DETAILED DESCRIPTION

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise. Such terms do not generally signify a closed list.

“Above,” “adhesive,” “affixing,” “any,” “around,” “both,” “bottom,” “by,” “comprising,” “consistent,” “customized,” “enclosing,” “friction,” “in,” “labeled,” “lower,” “magnetic,” “marked,” “new,” “nominal,” “not,” “of,” “other,” “outside,” “outwardly,” “particular,” “permanently,” “preventing,” “raised,” “respectively,” “reversibly,” “round,” “square,” “substantial,” “supporting,” “surrounded,” “surrounding,” “threaded,” “to,” “top,” “using,” “wherein,” “with,” or other such descriptors herein are used in their normal yes-or-no sense, not as terms of degree, unless context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to, or combined, without limiting the scope to the embodiments disclosed herein.

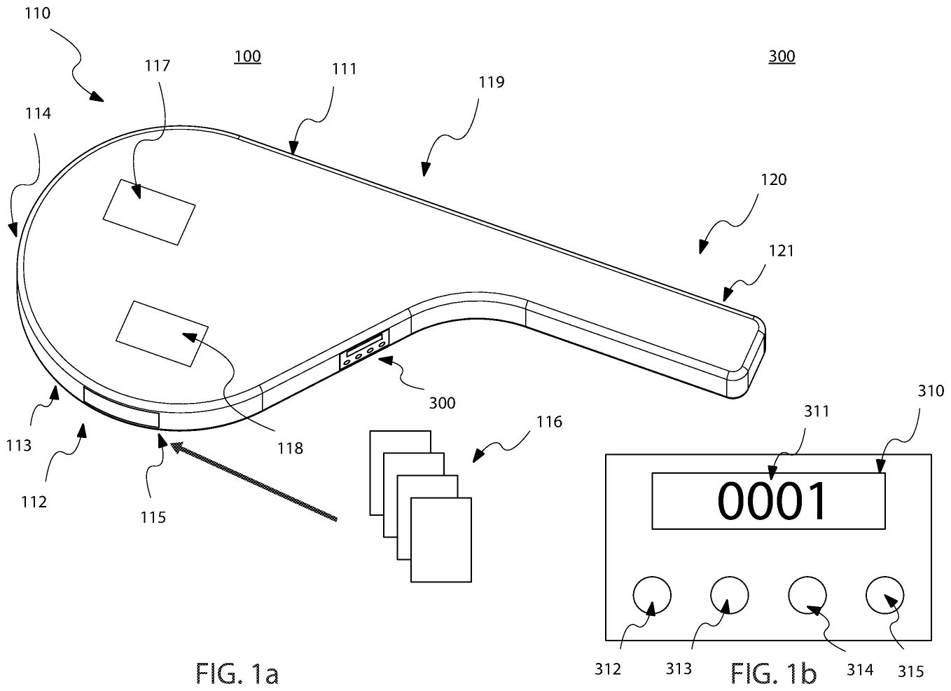

Referring to FIG. 1A and FIG. 1B, an illustrated view of an exemplary skull blocker 100 for protecting a person that has entered into a self-harming event is presented. The skull blocker 100 is useful for preventing injury to the person during a self-harming event and to diffuse or allow the event to conclude without harm to the person. Furthermore, the skull blocker 100 to be light weight, soft, reusable and easy to use. The skull blocker provides a calming effect and distracts the afflicted person from inflicting harm upon themselves. The skull blocker 100 further provides data for tracking impact events as well as reduces fatigue on any staff that is trying to hold or prevent the self-harming event.

The skull blocker 100 has a body 110 and a handle 120. The body 110 has a top 111, a bottom 112, a side 113, and an inside 114. The body 110 is preferably a round shape, however other shapes are hereby contemplated, including, but not limited to, square, guitar-like, oval, etc. The body 110 is preferably two (2) feet in diameter, however other diameters are hereby contemplated, including, but not limited to, one (1) foot, four (4) feet, etc. The body 110 is preferably two (2) feet in depth, however other depths are hereby contemplated, including, but not limited to, one and one-half (1.5) feet, two and one-half (2.5) feet, etc. An exterior 119 of the body 110 is preferably made of a soft material, however other materials are hereby contemplated, including, but not limited to, cotton with a bean-bag filling, denim with sand filling, etc. The side 113 of the body 110 preferably has one or more perforated facets 115, however it is hereby contemplated that there are no perforated facets 115 in the side 113 of the body 110. The perforated facets 115 are useful for providing a scented sheet 116 such that the skull blocker 100 emits a pleasant and calming fragrance.

The handle 120 is coupled to the body 110 of the skull blocker 100. The skull blocker 100 is preferably made of a single mold, however it is hereby contemplated that the handle 120 and the body 110 are made in separated mold and securely coupled for safety.

The handle 120 is predominately a rectangular shape, however other shapes are hereby contemplated, including, but not limited to, rectangular with a curved ending, oblong, etc. The handle 120 is preferably four (4) feet in length, however other lengths are hereby contemplated, including, but not limited to, three (3) feet, five (5) feet, etc. The handle 120 is preferably two (2) feet in depth, however other depths are hereby contemplated, including, but not limited to, one (1) foot, three (3) feet, etc. The handle 120 preferably has a depth the same as the depth of the body 110, however it is hereby contemplated that the depth of the handle 120 is not the same as the depth of the body 110.

An exterior 121 of the handle 120 is preferably made of a soft material, however other materials are hereby contemplated, including, but not limited to, cotton with a bean-bag filling, denim with sand filling, etc. The exterior 121 of the handle 120 and the exterior 119 of the body 110 may be made of the same material however it is hereby contemplated that the exterior 121 of the handle 120 and the exterior 119 of the body 110 may be made of the different materials.

The top 111 of the body 110 has one or more pressure pads 117, 118. The pressure pads 117, 118 are useful for reducing the force upon a strike of a hand, head, or other tool. The pressure pads 117, 118 may further be used to indicate the strike and increase a counter 311, such that the number of strikes is presented on a display panel 310 of a control panel 300.

The control panel 300 is coupled to the side 112 of the body 110, such that the display panel 310 and a plurality of audio signal devices 312, 313, 314, 315 are accessible by the person holding the skull blocker 100. The audio signal devices 312, 313, 314, 315 emit a sound, where each of the audio signal devices 312, 313, 314, 315 provide a unique sound or volume level of the unique sound. The sound is preferably an alarm, but may be any sound that may alert, comfort, or signal a distress condition to others.

In the numbered clauses below, specific combinations of aspects and embodiments are articulated in a shorthand form such that (1) according to respective embodiments, for each instance in which a “component” or other such identifiers appear to be introduced (with “a” or “an,” e.g.) more than once in a given chain of clauses, such designations may either identify the same entity or distinct entities; and (2) what might be called “dependent” clauses below may or may not incorporate, in respective embodiments, the features of “independent” clauses to which they refer or other features described above.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A skull blocker for protecting a person that has entered into a self-harming event, the skull blocker comprising:
a body, the body comprising:
a top, the top having one or more pressure pads, the pressure pads being for providing a shock reduction of a strike;
a back;
a side;
a control panel, the control panel being coupled to the side, wherein the control panel having a counter and a plurality of audio signal devices, wherein the counter being increased by sensing of a strike by one or more of the pressure pads; and
a handle, the handle being coupled to the body, wherein the handle extending through the body.

2. The skull blocker of claim 1, wherein an exterior of the body being made of a foam material.

3. The skull blocker of claim 1, wherein the body have a round shape.

4. The skull blocker of claim 1, wherein the handle having a length of four (4) feet.

5. The skull blocker of claim 1, wherein the body having a diameter of two (2) feet.

6. The skull blocker of claim 1, wherein the body having a depth of two (2) feet.

7. The skull blocker of claim 1, wherein the pressure pads sense a strike and increase the counter.

8. The skull blocker of claim 1, wherein an exterior of the body being made of a soft material.

9. The skull blocker of claim 1, wherein an exterior of the handle being made of a soft material.

10. The skull blocker of claim 1, wherein an exterior of the handle and an exterior of the body being made of a same material.

11. The skull blocker of claim 1, wherein the side of the body having one or more perforated facets, wherein each of the perforated facets being configured to receive a scent.
